(19) United States
(12) Patent Application Publication
(10) Pub. No.: US 2025/0303418 A1
(43) Pub. Date: Oct. 2, 2025

(54) CELL PUNCTURE DEVICE AND MICROSCOPE SYSTEM

(71) Applicant: Yokogawa Electric Corporation, Tokyo (JP)

(72) Inventor: Masahiro KUWATA, Tokyo (JP)

(73) Assignee: Yokogawa Electric Corporation, Tokyo (JP)

(21) Appl. No.: 19/087,730

(22) Filed: Mar. 24, 2025

(30) Foreign Application Priority Data
Mar. 29, 2024 (JP) 2024-057948

(51) Int. Cl. B01L 3/00; G02B 21/00; H04N 23/68

(52) U.S. Cl. CPC B01L 3/502761; H04N 23/682; B01L 2200/0647; B01L 2300/0609; B01L 2300/0672; G02B 21/0024

(57) ABSTRACT

A cell puncture device 10 according to the present disclosure includes a first fixed part 11 arranged with respect to an imaging unit 21 configured to image a cell S, an arm 14 arranged with respect to the first fixed part 11, a second driver 16 arranged with respect to the arm 14, a needle 17a that is configured to be driven by the second driver 16 and to puncture the cell S, and vibration dampers 19a configured to dampen vibration of the needle 17a. The vibration dampers 19a are arranged with respect to at least one of the arm 14 or the second driver 16.

### CROSS-REFERENCE TO RELATED APPLICATION

This application claims priority to Japanese Patent Application No. 2024-057948, filed on Mar. 29, 2024, the entire contents of which are incorporated herein by reference.

### TECHNICAL FIELD

The present disclosure relates to a cell puncture device and a microscope system.

### BACKGROUND

In conventional research and applied development related to cells, technology for puncturing cells with needles is known, in order to accurately inject chemical solutions or the like into specific cells as samples, or to suck substances inside cells. For example, Patent Literature (PTL) 1 discloses that, in a device that uses a multi-barrel nano-pipette with at least two electrodes inside multiple barrels, one barrel draws out a cell inclusion and the other injects a substance into a cell.

### CITATION LIST

### Patent Literature

PTL 1: JP 6453300 B2

### SUMMARY

A cell puncture device according to some embodiments includes:
a first fixed part arranged with respect to an imaging unit configured to image a cell;
an arm arranged with respect to the first fixed part;
a driver arranged with respect to the arm;
a needle configured to be driven by the driver, the needle configured to puncture the cell; and
a vibration damper configured to dampen vibration of the needle,
wherein the vibration damper is arranged with respect to at least one of the arm or the driver.

### BRIEF DESCRIPTION OF THE DRAWINGS

In the accompanying drawings:

FIG. 1 is a schematic diagram illustrating an example configuration of a microscope system with a cell puncture device according to a first embodiment of the present disclosure;

FIG. 2 is a schematic diagram illustrating an example configuration of a microscope system with a cell puncture device according to a first variation of the first embodiment of the present disclosure;

FIG. 3 is a schematic diagram illustrating an example configuration of a microscope system with a cell puncture device according to a second variation of the first embodiment of the present disclosure;

FIG. 4 is a schematic diagram illustrating an example configuration of a microscope system with a cell puncture device according to a second embodiment of the present disclosure; and

FIG. 5 is a graph for explaining an effect based on the cell puncture device in FIG. 1.

### DETAILED DESCRIPTION

In the conventional technology described in PTL 1, for example, when a needle punctures a cell, a needle tip of the needle may vibrate in the interior of a cell wall and cause damage to the interior of the cell wall. When vibration is large, it is assumed that the needle is repeatedly inserted into and removed from the cell, which also causes damage to the cell.

It would be helpful to provide a cell puncture device and a microscope system that can suppress vibration of a needle.

A cell puncture device according to some embodiments includes:
a first fixed part arranged with respect to an imaging unit configured to image a cell;
an arm arranged with respect to the first fixed part;
a driver arranged with respect to the arm;
a needle configured to be driven by the driver, the needle configured to puncture the cell; and
a vibration damper configured to dampen vibration of the needle,
wherein the vibration damper is arranged with respect to at least one of the arm or the driver.

Therefore, the cell puncture device can suppress the vibration of the needle. For example, by arranging the vibration damper with respect to the driver, the cell puncture device can also suppress the vibration of the needle based on a reaction force when the driver drives the needle. For example, the cell puncture device can suppress the vibration that occurs due to a reaction force on a fixed part's side of the driver when part of the driver moves to puncture the cell with the needle. For example, the cell puncture device can also suppress the vibration of the needle based on a force of reaction when the needle punctures the cell. For example, the cell puncture device can even suppress the vibration of the needle due to some external factor that is subjected while the driver is not driving the needle.

The cell puncture device can suppress the vibration of the needle connected to the driver, and therefore reduce damage to the interior of the cell wall and to the cell. By arranging the vibration damper with respect to the arm, the cell puncture device can suppress vibration of the entire structure, and therefore suppress the vibration of the needle. The cell puncture device can suppress the vibration of the needle even when the needle is moved at a high speed to penetrate the cell wall and puncture the interior of the cell and the needle after the puncture vibrates due to a reaction force associated with the high-speed movement of the driver. Therefore, even when a chemical solution is injected into the cell and then the subsequent progress is observed, for example, the cell puncture device can suppress damage to the cell caused by the vibration of the needle tip, thus suppressing a reduction in a survival rate of the cell. Therefore, the cell puncture device can contribute to accurate evaluation of the effects of the injected chemical solution.

In the cell puncture device according to one embodiment, the vibration damper may be arranged such that a vibration-damping surface intersects a puncture operation direction of the needle. This allows the cell puncture device to more effectively dampen the vibration of the needle after the puncture in the puncture operation direction. In other words, the cell puncture device can absorb, using the vibration damper, more vibration energy than when the vibration-damping surface is arranged in parallel with the puncture operation direction of the needle.

The cell puncture device according to one embodiment may further include a regulator arranged with respect to the driver, the regulator configured to regulate a movement direction of the driver to the puncture operation direction of the needle. This allows the cell puncture device to reduce fluctuations in the position of the needle in other directions that differ from the puncture operation direction. Therefore, in a case in which the cell puncture device accurately controls the position of the needle using the driver to match a desired position for the cell, even when the needle is subjected to vibration due to some external factor, the cell puncture device can reduce the movement of the needle in other directions that differ from the puncture operation direction. In addition, the cell puncture device can also suppress the vibration of the needle more effectively by regulating the movement direction of the driver to only the puncture operation direction of the needle.

In the cell puncture device according to one embodiment, the regulator may include:
a third fixed part arranged with respect to the arm; and
a second movable part connected to the third fixed part and the driver.

This allows the cell puncture device to regulate the movement direction of the driver to the puncture operation direction of the needle, as described above. In addition, by regulating the movement direction of the driver to the puncture operation direction, for example, the cell puncture device can receive, at the third fixed part via the second movable part, vibration that occurs in the driver in directions other than the puncture operation direction. Therefore, the cell puncture device can reduce such vibration using the third fixed part. In the cell puncture device, the third fixed part is arranged with respect to the second movable part, and the second movable part is movable only in the puncture operation direction with respect to the third fixed part, which is fixed. The cell puncture device can reduce, using the third fixed part, vibration in the directions other than the puncture operation direction, and also reduce vibration transmitted to a support, the arm, and the like. Therefore, the cell puncture device can prevent the entire cell puncture device from vibrating.

In the cell puncture device according to one embodiment,
the driver may include:
   a second fixed part connected to the regulator; and
   a first movable part connected to the second fixed part, the first movable part configured to drive the needle, and
the vibration damper may be arranged such that a vibration-damping surface contacts only the first movable part.

This allows the cell puncture device to effectively suppress the vibration of the needle, by arranging the vibration damper on the first movable part, to which a needle unit is connected and which is directly affected by the vibration of the needle.

In the cell puncture device according to one embodiment,
the driver may include:
   a second fixed part connected to the regulator; and
   a first movable part connected to the second fixed part, the first movable part configured to drive the needle, and
the vibration damper may be arranged such that a vibration-damping surface contacts both the second fixed part and the first movable part.

This allows the cell puncture device to receive, using the vibration damper, the vibration of the needle in a wide range that includes not only the first movable part but also the second fixed part. Therefore, the cell puncture device can dampen the vibration of the needle more effectively.

In the cell puncture device according to one embodiment,
the driver may include:
   a second fixed part connected to the regulator; and
   a first movable part connected to the second fixed part, the first movable part configured to drive the needle, and
the vibration damper may be arranged such that a vibration-damping surface contacts only the second fixed part.

This allows the cell puncture device to eliminate the need for arranging the support, which is described later, around the first movable part, and improve flexibility in movement of the first movable part. Therefore, even when a microscope is arranged close to the cell puncture device and space for arranging the vibration damper is limited, the vibration damper can be easily arranged within that space in the cell puncture device.

In addition, the cell puncture device can cause the first movable part to move quickly because the first movable part does not receive a reaction force corresponding to deformation of the vibration damper, as compared to when the vibration damper is arranged with respect to the first movable part. This allows the cell puncture device to achieve high-speed movement of the first movable part when the needle moves at a high speed and punctures the cell. The cell puncture device can reduce the weight of an actuator, e.g., a piezoelectric element contained in part of the first movable part and reduce a reaction force due to movement of the first movable part, as compared to the case of increasing the size of the piezoelectric element, for example, so that a large force for movement can be obtained in the first movable part when the first movable part receives the reaction force corresponding to the deformation of the vibration damper. As a result, vibration occurring by the reaction force is reduced.

In the cell puncture device according to one embodiment, the vibration damper may be arranged such that a vibration-damping surface contacts the arm. This allows the cell puncture device to eliminate the need for arranging the support, which is described later, around the entire driver, and therefore improve the flexibility of movement of the driver. Therefore, the cell puncture device can suppress the vibration of the needle by arranging the vibration damper on the arm even when the microscope is located near the cell puncture device and the vibration damper is difficult to arrange around the driver. Also, the cell puncture device can dampen vibration that is transmitted to the arm, and therefore can suppress vibration that is transmitted to the entire cell puncture device more effectively.

In the cell puncture device according to one embodiment, the driver may include a piezoelectric element configured to drive the needle. This allows the cell puncture device to move the needle at a high speed when the needle punctures the cell. Therefore, the cell puncture device can easily control the position and speed of the needle tip so that the needle tip penetrates the cell wall in order to inject a chemical solution into the cell or to take out a substance from the interior of the cell by suction. For example, when the needle punctures a plant cell with a hard cell wall, the cell puncture device can cause the needle to puncture the cell at a speed sufficient to penetrate the cell wall.

In the cell puncture device according to one embodiment, the vibration damper may include vibration-damping rubber. This allows the cell puncture device to convert part of vibration energy into thermal energy using the vibration damper being pressed and deformed by the vibration of the needle, thus allowing the vibration energy to be efficiently dampened and the vibration to be suppressed more effectively.

In a microscope system according to some embodiments includes:
the cell puncture device according to any of the above; and
a microscope having the imaging unit configured to image the cell to be punctured by the needle of the cell puncture device.

Therefore, the microscope system can suppress the vibration of the needle. For example, in the microscope system, the microscope is arranged with respect to the first fixed part, and the cell puncture device dampens, using the vibration damper, vibration that occurs due to a reaction force on the fixed part's side of the driver when part of the driver moves to puncture the cell on the microscope with the needle. Therefore, the microscope system reduces damage to the cell and eases observation of the cell punctured by the needle.

According to the present disclosure, it is possible to provide the cell puncture device and the microscope system that can suppress the vibration of the needle.

The background and problems of conventional technology will be described in more detail.

In recent years, in research into biological systems and the like, there have been research into elucidating cell functions by injecting specific chemical solutions into cells and observing changes in the cells, and research into causing specific modifications to occur in specific cells by injecting, into the cells, chemical solutions to modify genes. In addition, there has also been applied development aiming at application to the production of chemical solutions or other substances. On the other hand, there have also been research into elucidating cell functions and applied development aiming at application to production, by sucking and recovering, from specific cells, some of components that constitute the cells. In the above research and applied development, it is required, for example, to accurately inject the chemical solutions into the specific cells or to accurately suck the components from the specific cells, so it is desired that cell puncture devices can accurately puncture cells with needles.

PTL 1 discloses conventional technology for a method and device of injecting a chemical solution into a cell by controlling the position of a fine needle using a piezoelectric element, puncturing the cell by a needle tip, and controlling a voltage. Similarly, conventional technology for a method and device for sucking a substance from the interior of the cell is also disclosed.

In general, cells protect their interiors by cell walls, which are located in more external parts of the cells and cover the interiors. Therefore, in order to inject a chemical solution into a cell or to take out a substance from the interior of a cell by suction, a cell puncture device needs to insert a needle into the interior of the cell by controlling the position and speed of a needle tip so that the needle tip penetrates a cell wall. For this purpose, the cell puncture device preferably moves the needle at a high speed when the needle punctures the cell.

For example, in the case of a certain animal cell with a soft cell wall, the needle can penetrate the cell wall even if the cell puncture device moves the needle at a low speed when the needle punctures the cell. On the other hand, in the case of a certain plant cell with a hard cell wall, the needle cannot penetrate the cell wall unless the cell puncture device moves the needle at a high speed when the needle punctures the cell, which causes the problem of difficulty in inserting the needle tip into the cell.

As a method of moving the needle at a high speed as described above, a method of moving the needle using a piezoelectric element included in a driver that drives the needle is known. The piezoelectric element can cause an electrostrictive effect to occur inside itself by application of a voltage, and can expand and contract the piezoelectric element itself. The expansion and contraction of the piezoelectric element can respond at a higher speed than actuators such as general motors. Therefore, with the use of the piezoelectric element, the cell puncture device can cause the needle to puncture the cell at a high speed.

However, due to the high-speed movement of the needle using the piezoelectric element, there is a problem that the needle vibrates based on a reaction force when the needle is driven by the driver. For example, when part of the driver moves to puncture the cell, a reaction force occurs on a fixed side of the driver and causes vibration. In addition, when the needle punctures the cell, a force due to reaction is applied to a structure that supports the piezoelectric element. This causes the entire structure to move in the opposite direction to a puncture operation direction of the needle. As a result, there is also a problem that the needle vibrates after the puncture. When the needle vibrates after the puncture, the needle tip may vibrate in the interior of the cell wall, and cause damage to the interior of the cell wall. When the vibration is large, it is assumed that the needle is repeatedly inserted into and removed from the cell, which causes damage to the cell. Therefore, for example, when a chemical solution is injected into a cell and its progress is observed, a cell survival rate is reduced due to damage to the cell caused by the vibration of the needle tip. As a result, there are cases in which the effects of the injected chemical solution cannot be evaluated.

In order to solve the problems described above, it would be helpful to provide a cell puncture device and a microscope system that can suppress vibration of a needle.

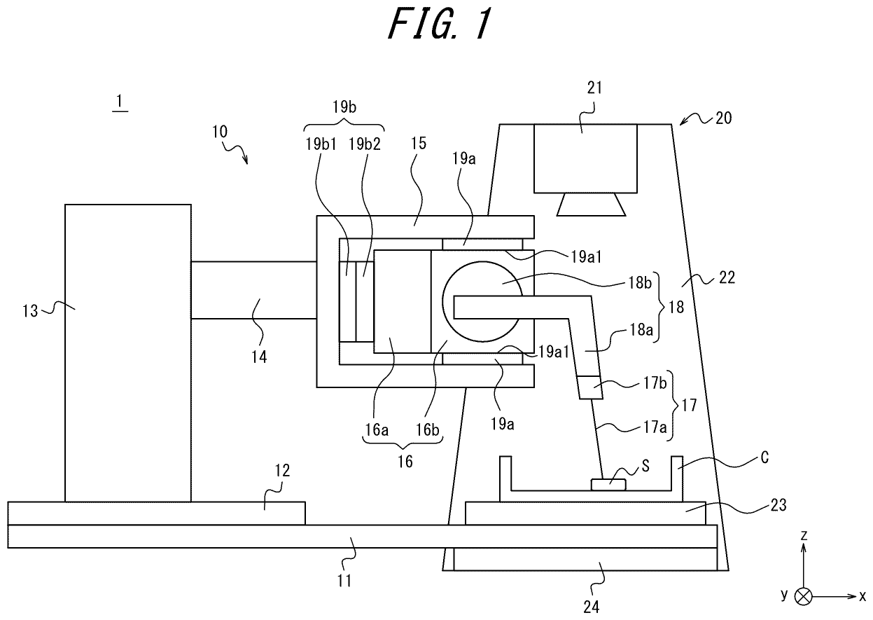

Embodiments of the present disclosure will be mainly described below with reference to the attached drawings. In the following description, x-, y-, and z-directions are with respect to the directions of the arrows in the drawings. The directions of the arrows are consistent with each other in different drawings in FIGS. 1 to 4.

### First Embodiment

FIG. 1 is a schematic diagram illustrating an example configuration of a microscope system 1 having a cell puncture device 10 according to a first embodiment of the present disclosure. An example of the configuration and functions of the microscope system 1 having the cell puncture device 10 according to the first embodiment will be mainly described with reference to FIG. 1. The microscope system 1 has the cell puncture device 10 and a microscope 20 that images a cell S to be punctured by a needle 17a of the cell puncture device 10.

The microscope 20 includes any microscope that can image the cell S. The microscope 20 includes, for example, a confocal microscope. The microscope 20 has any camera 21 that can image the cell S. The camera 21 constitutes an imaging unit of the microscope 20. The microscope 20 has a pillar 22 that locates the camera 21 on one side in the z-direction with respect to the cell S, so that the camera 21 can image the cell S from that side. The pillar 22 supports the camera 21 connected to an end of the pillar 22 on that side. The microscope 20 has a holder 23 that holds a petri dish C, on which the cell S is disposed, from the other side in the z-direction. The holder 23 is configured as a stage that is movable in two directions, the x- and y-directions. The microscope 20 has a support 24 that is located at an end of the pillar 22 on the other side and that supports the holder 23, which holds the petri dish C.

The cell puncture device 10 has a first fixed part 11 that is arranged with respect to the imaging unit, which images the cell S. The first fixed part 11 is, for example, fixed to the microscope 20, which images the cell S. The first fixed part 11 is configured in the shape of an arm and extends in the x-direction. The first fixed part 11 is arranged with respect to the holder 23 and the support 24 and is fixed to the microscope 20, by being screwed at one side in the x-direction onto the support 24 of the microscope 20. As an example, the first fixed part 11 is located between the holder 23 and the support 24, but is not limited to this. The holder 23 may be located under the first fixed part 11. The first fixed part 11 is not limited to being screwed onto the support 24, but may be fixed to the microscope 20 in any other manner, such as by joining, fitting, or engaging. The cell puncture device 10 can be attached to the microscope 20 via the first fixed part 11.

The cell puncture device 10 is connected to the other side of the first fixed part 11 in the x-direction, and has a pedestal 12 located on a surface of the first fixed part 11. The cell puncture device 10 is supported by the first fixed part 11 and the pedestal 12, and has a first driver 13 that protrudes from the pedestal 12 to the positive side in the z-direction. The cell puncture device 10 has an arm 14 that extends from the first driver 13 to the positive side in the x-direction. The arm 14 is arranged with respect to the first fixed part 11. For example, the arm 14 is arranged in parallel with the first fixed part 11. The first driver 13 drives the arm 14 so that the arm 14 is movable in each of the x-, y-, and z-directions with respect to the pedestal 12.

The cell puncture device 10 has a support 15 that is connected to a distal end of the arm 14 in the x-direction. The support 15 is for mounting vibration dampers 19a and a regulator 19b, which are described later. The cell puncture device 10 has a second driver 16 located inside the support 15 so as to be sandwiched by an outer frame of the support 15. Not limited to this, the second driver 16 may not be located inside the support 15. The second driver 16 may be located in another place outside the support 15, together with the vibration dampers 19a. For example, the vibration dampers 19a may be attached to another place that is not related to the support 15, e.g., an exterior of the cell puncture device 10, and the second driver 16. The second driver 16 is arranged with respect to the arm 14 via the regulator 19b and the support 15. The second driver 16 is arranged with respect to the support 15 via the vibration dampers 19a.

The second driver 16 needs to move the needle 17a at a high speed in order to puncture the cell S. In order to achieve such high-speed movement of the needle 17a, the second driver 16 includes, for example, a piezoelectric element that drives the needle 17a. The second driver 16 includes a second fixed part 16a that is connected to the regulator 19b, which is described later, and a first movable part 16b that is connected to the second fixed part 16a and drives the needle 17a. The first movable part 16b is movable relative to the second fixed part 16a. The piezoelectric element contained in the first movable part 16b drives the needle 17a so that a needle tip of the needle 17a moves along the z-direction. The first movable part 16b causes the needle 17a to puncture the cell S by moving relative to the second fixed part 16a.

The cell puncture device 10 has a needle unit 17 that arranges, at a tip end, the needle 17a to puncture the cell S and that is driven by the first movable part 16b of the second driver 16. The needle unit 17 has the needle 17a that is driven by the second driver 16 and punctures the cell S, and a needle fixer 17b that fixes the needle 17a.

The cell puncture device 10 has a needle support 18 that is connected to the needle fixer 17b and that arranges the needle unit 17 at its distal end. The needle support 18 has a support head 18a connected to the needle fixer 17b, and a support head fixer 18b connected to the support head 18a. The needle unit 17 can be operated by the second driver 16, by being supported by the support head 18a and by the support head 18a being attached to the support head fixer 18b.

The cell puncture device 10 has the vibration dampers 19a that dampen vibration of the needle 17a. The vibration dampers 19a include, for example, vibration-damping rubber. The vibration dampers 19a may be arranged with respect to at least one of the arm 14 or the second driver 16. For example, in FIG. 1, the vibration dampers 19a are arranged so that vibration-damping surfaces 19al contact only the first movable part 16b of the second driver 16. The pair of vibration dampers 19a is arranged on both sides of the second driver 16 in the z-direction. Not limited to this, only one vibration damper 19a may be arranged with respect to the second driver 16, or three or more vibration dampers 19a may be arranged. The vibration dampers 19a may be arranged between the support 15 and the second driver 16 so as to fill gaps between the support 15 and the second driver 16 along the z-direction.

The vibration dampers 19a are arranged so that the vibration-damping surfaces 19al intersect a puncture operation direction of the needle 17a. In the present disclosure, the “puncture operation direction” corresponds to, for example, the z-direction. The phrase of “intersect a puncture operation direction” means not only intersecting perpendicularly to the puncture operation direction, but also intersecting inclinedly to the puncture operation direction. For example, the vibration-damping surfaces 19al are orthogonal to the z-direction, which is the puncture operation direction of the needle 17a. The vibration-damping surfaces 19al constitute contact surfaces with the first movable part 16b in the vibration dampers 19a, and contact surfaces of the first movable part 16b in the z-direction. The vibration-damping surfaces 19al are included in the xy plane, as an example. When the second driver 16 vibrates and deforms the vibration dampers 19a by pressing the vibration dampers 19a, the vibration dampers 19a dampen the vibration by converting part of vibration energy of the second driver 16 into thermal energy.

The cell puncture device 10 has the regulator 19b that is arranged with respect to the second driver 16 and that regulates a movement direction of the second driver 16 to the puncture operation direction of the needle 17a. The regulator 19b includes a third fixed part 19b1 that is arranged with respect to the arm 14, and a second movable part 19b2 that is connected to the third fixed part 19b1 and the second fixed part 16a of the second driver 16. The third fixed part 19b1 is connected to, for example, an inner surface of the support 15 along the z-direction.

The regulator 19b includes, for example, a linear guide, a cross roller guide, or the like. The regulator 19b allows the second driver 16 to move in the puncture operation direction to cause the needle 17a to puncture the cell S, but regulates the movement of the second driver 16 in directions orthogonal to the puncture operation direction. For example, when the puncture operation direction is the z-direction, the regulator 19b allows the movement of the second driver 16 along the z-direction, but regulates the movement of the second driver 16 in the x- and y-directions.

The cell puncture device 10 may be configured so that the entire structure, which includes the first driver 13, the arm 14, the support 15, the second driver 16, and the needle 17a, can be evacuated from the microscope 20, by moving the pedestal 12 in the x- or y-direction relative to the first fixed part 11 or by rotatably moving the pedestal 12. The movement of the pedestal 12 in the x- or y-direction can be easily achieved by, for example, arranging a linear guide, a cross roller guide, or the like between the first fixed part 11 and the pedestal 12. The rotational movement of the pedestal 12 can be easily achieved by, for example, arranging a ball bearing, a cross roller bearing, or the like between the first fixed part 11 and the pedestal 12.

The cell puncture device 10 may further have a fixed part (not illustrated in the drawings) between the first fixed part 11 and the pedestal 12 in order to fix the relative movement of the pedestal 12 with respect to the first fixed part 11 in the x- or y-direction, or the relative rotational movement. For example, the fixed part may fix the pedestal 12 by a frictional force of a pin that can move in the z-direction and is pressed against the first fixed part 11 by a spring, or may fix the pedestal 12 by a similar pin engaging a groove provided in the first fixed part 11. The pin can be easily lifted up by arranging an operation unit to lift up the pin to the positive side in the z-direction. The pin allows the pedestal 12 to be moved and fixed relative to the first fixed part 11.

The cell puncture device 10 can accurately arrange the pedestal 12 with respect to the first fixed part 11, due to the above-described configuration. The cell puncture device 10 can reduce fluctuations in the position of the needle 17a due to play of the pedestal 12 with respect to the first fixed part 11.

The cell puncture device 10 and the microscope system 1 according to the first embodiment as described above can suppress the vibration of the needle 17a. For example, by arranging the vibration dampers 19a with respect to the second driver 16, the cell puncture device 10 can also suppress the vibration of the needle 17a based on a reaction force when the second driver 16 drives the needle 17a. For example, the cell puncture device 10 can suppress the vibration that occurs due to a reaction force on the fixed part's side of the second driver 16 when part of the second driver 16 moves to puncture the cell S with the needle 17a. For example, the cell puncture device 10 can also suppress the vibration of the needle 17a based on a force of reaction when the needle 17a punctures the cell S. For example, the cell puncture device 10 can even suppress the vibration of the needle 17a due to some external factor that is subjected while the second driver 16 is not driving the needle 17a.

The cell puncture device 10 can suppress the vibration of the needle 17a connected to the second driver 16, and therefore reduce damage to the interior of a cell wall of the cell S and to the cell S. The cell puncture device 10 can suppress the vibration of the needle 17a even when the needle 17a is moved at a high speed to penetrate the cell wall of the cell S and puncture the interior of the cell S and the needle 17a after the puncture vibrates due to a reaction force associated with the high-speed movement of the second driver 16. Therefore, for example, even when a chemical solution is injected into the cell S and then the subsequent progress is observed, the cell puncture device 10 can suppress damage to the cell S caused by the vibration of the needle tip of the needle 17a, thus suppressing a reduction in a survival rate of the cell S. Therefore, the cell puncture device 10 can contribute to accurate evaluation of the effects of the injected chemical solution.

In the cell puncture device 10, the vibration dampers 19a are arranged such that the vibration-damping surfaces 19al intersect the puncture operation direction of the needle 17a. This allows the cell puncture device 10 to more effectively dampen the vibration of the needle 17a after the puncture in the puncture operation direction. In other words, the cell puncture device 10 can absorb, using the vibration dampers 19a, more vibration energy than when the vibration-damping surfaces 19al are arranged in parallel with the puncture operation direction of the needle 17a.

The cell puncture device 10 has the regulator 19b that is arranged with respect to the second driver 16 and that regulates a movement direction of the second driver 16 to the puncture operation direction of the needle 17a. This allows the cell puncture device 10 to reduce fluctuations in the position of the needle 17a in other directions that differ from the puncture operation direction. Therefore, in a case in which the cell puncture device 10 accurately controls the position of the needle 17a using the second driver 16 to match a desired position for the cell S, even when the needle 17a is subjected to vibration due to some external factor, the cell puncture device 10 can reduce the movement of the needle 17a in other directions that differ from the puncture operation direction. In addition, the cell puncture device 10 can also suppress the vibration of the needle 17a more effectively by regulating the movement direction of the second driver 16 to only the puncture operation direction of the needle 17a.

In the cell puncture device 10, the regulator 19b includes a third fixed part 19b1 that is arranged with respect to the arm 14, and a second movable part 19b2 that is connected to the third fixed part 19b1 and the second driver 16. This allows the cell puncture device 10 to regulate the movement direction of the second driver 16 to the puncture operation direction of the needle 17a, as described above. In addition, by regulating the movement direction of the second driver 16 to the puncture operation direction, the cell puncture device 10 can receive, at the third fixed part 19b1 via the second movable part 19b2, vibration that occurs in the second driver 16 in directions other than the puncture operation direction. Therefore, the cell puncture device 10 can reduce such vibration using the third fixed part 19b1. In the cell puncture device 10, the third fixed part 19b1 is arranged with respect to the second movable part 19b2, and the second movable part 19b2 is movable only in the puncture operation direction with respect to the third fixed part 19b1, which is fixed. The cell puncture device 10 can reduce, using the third fixed part 19b1, vibration in the directions other than the puncture operation direction, and also reduce vibration transmitted to the support 15, the arm 14, and the like. Therefore, the cell puncture device 10 can prevent the entire cell puncture device 10 from vibrating.

In the cell puncture device 10, the vibration dampers 19a may be arranged so that the vibration-damping surfaces 19al contact only the first movable part 16b. This allows the cell puncture device 10 to effectively suppress the vibration of the needle 17a, by arranging the vibration dampers 19a on the first movable part 16b, to which the needle unit 17 is connected and which is directly affected by the vibration of the needle 17a.

In the cell puncture device 10, the second driver 16 includes the piezoelectric element that drives the needle 17a. This allows the cell puncture device 10 to move the needle 17a at a high speed when the needle 17a punctures the cell S. Therefore, the cell puncture device 10 can easily control the position and speed of the needle tip so that the needle tip penetrates the cell wall of the cell S in order to inject a chemical solution into the cell S or to take out a substance from the interior of the cell S by suction. For example, when the needle 17a punctures a plant cell with a hard cell wall, the cell puncture device 10 can cause the needle 17a to puncture the cell at a speed sufficient to penetrate the cell wall.

In the cell puncture device 10, the vibration dampers 19a may include the vibration-damping rubber. This allows the cell puncture device 10 to convert part of the vibration energy into thermal energy using the vibration dampers 19a being pressed and deformed by the vibration of the needle 17a, thus allowing the vibration energy to be efficiently dampened and the vibration to be suppressed more effectively.

As described above, in a case in which the needle 17a punctures the cell S using the cell puncture device 10, it is necessary to place the vibration dampers 19a as illustrated in FIG. 1 in order to dampen the vibration of the needle 17a based on the reaction force when the first movable part 16b of the second driver 16 moves. Suppressing the vibration of the needle 17a after the puncture reduces damage to the cell S.

For example, the cell puncture device 10 can effectively dampen the vibration by arranging the vibration dampers 19a such that the vibration-damping surfaces 19al contact the first movable part 16b of the second driver 16 connected to the needle 17a, which is a source of the vibration. By arranging the vibration dampers 19a in positions close to the needle 17a, vibration transmitted to the vibration dampers 19a has an almost single period and is therefore easily damped by the vibration dampers 19a.

In the first embodiment described above, for example, the first fixed part 11 is described as being fixed to the microscope 20 that images the cell S, but is not limited to this. The first fixed part 11 may be attached to the microscope 20 in another aspect, as long as the first fixed part 11 is arranged with respect to the imaging unit that images the cell S. For example, the first fixed part 11 may be incorporated inside the microscope 20 or may be incorporated inside an inspection system in which the microscope 20 is incorporated, instead of being fixed so as to extend outside the microscope 20 as described above.

In the first embodiment described above, the vibration dampers 19a are described as being arranged such that the vibration-damping surfaces 19a1 intersect the puncture operation direction of the needle 17a, but is not limited to this. The vibration dampers 19a may be arranged such that the vibration-damping surfaces 19al are in parallel with the puncture operation direction of the needle 17a. Alternatively, the vibration dampers 19a may be arranged such that one vibration-damping surface 19al is orthogonal to the puncture operation direction of the needle 17a, and the other vibration-damping surface 19al is arranged in parallel with the puncture operation direction of the needle 17a.

In the first embodiment described above, the cell puncture device 10 is described as further having the regulator 19b that is arranged with respect to the second driver 16 and that regulates the movement direction of the second driver 16 to the puncture operation direction of the needle 17a, but is not limited to this. The regulator 19b may regulate the movement direction of the second driver 16 to a direction different from the puncture operation direction of the needle 17a. In addition, the cell puncture device 10 may not have the regulator 19b.

In the first embodiment described above, the second driver 16 is described as including the second fixed part 16a connected to the regulator 19b, and the first movable part 16b that is connected to the second fixed part 16a and that drives the needle 17a, but is not limited to this. The second driver 16 may be divided into three or more by fixed and movable parts, or may be integral without being divided into fixed and movable parts.

In the first embodiment described above, the regulator 19b is described as including the third fixed part 19b1 arranged with respect to the arm 14 and the second movable part 19b2 connected to the third fixed part 19b1 and the second driver 16, but is not limited to this. The regulator 19b may be divided into three or more by fixed and movable parts, or may be integral without being divided into fixed and movable parts.

In the first embodiment described above, the vibration dampers 19a are described as being arranged such that the vibration-damping surfaces 19al contact only the first movable part 16b of the second driver 16, but are not limited to this.

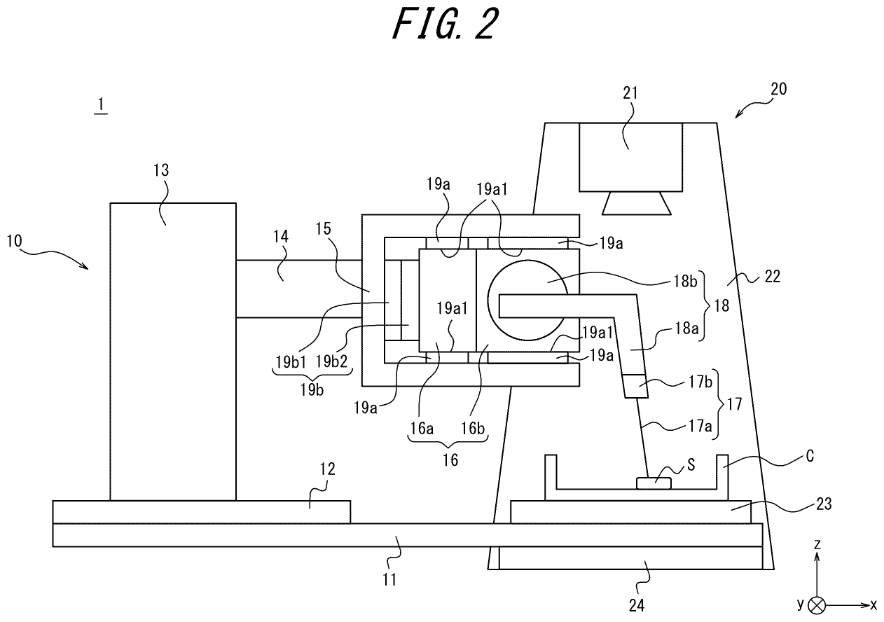

FIG. 2 is a schematic diagram illustrating an example configuration of a microscope system 1 with a cell puncture device 10 according to a first variation of the first embodiment of the present disclosure. As illustrated in FIG. 2, the vibration dampers 19a may be arranged such that the vibration-damping surfaces 19al contact both the second fixed part 16a and the first movable part 16b of the second driver 16.

This allows the cell puncture device 10 to receive, using the vibration dampers 19a, the vibration of the needle 17a in a wide range that includes not only the first movable part 16b but also the second fixed part 16a. Therefore, the cell puncture device 10 can dampen the vibration of the needle 17a more effectively.

In the first variation described above, as illustrated in FIG. 2, the vibration dampers 19a are arranged with being divided in the x-direction along the second fixed part 16a and the first movable part 16b, but are not limited to this. The vibration dampers 19a may be arranged integrally in the x-direction along the second fixed part 16a and the first movable part 16b.

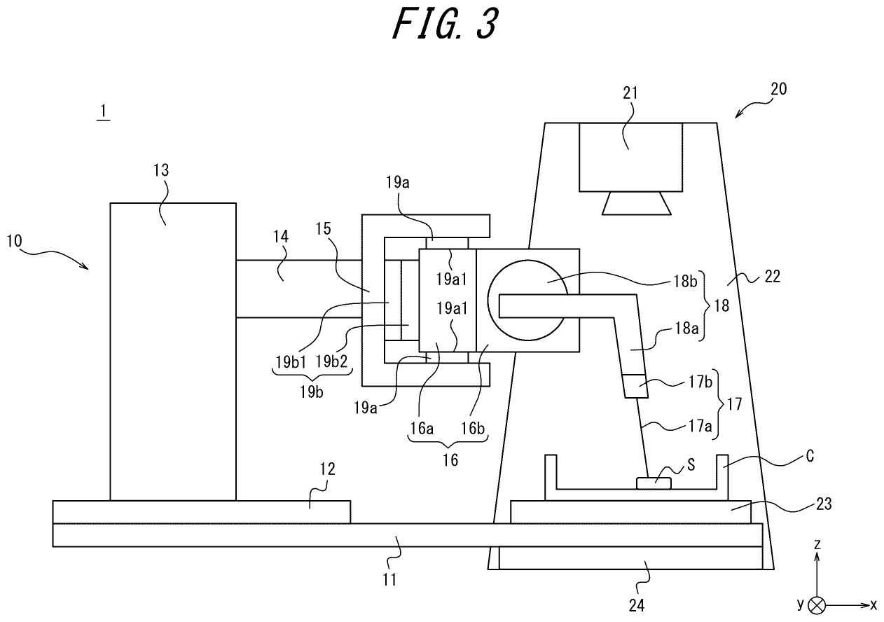

FIG. 3 is a schematic diagram illustrating an example configuration of a microscope system 1 with a cell puncture device 10 according to a second variation of the first embodiment of the present disclosure. As illustrated in FIG. 3, the vibration dampers 19a may be arranged such that the vibration-damping surfaces 19al contact only the second fixed part 16a of the second driver 16.

This allows the cell puncture device 10 to eliminate the need for arranging the support 15 around the first movable part 16b, and improve flexibility in movement of the first movable part 16b. Therefore, even when the microscope 20 is arranged close to the cell puncture device 10 and space for arranging the vibration dampers 19a is limited, the vibration dampers 19a can be easily arranged within that space in the cell puncture device 10.

In addition, the cell puncture device 10 can cause the first movable part 16b to move quickly because the first movable part 16b does not receive a reaction force corresponding to deformation of the vibration dampers 19a, as compared to when the vibration dampers 19a are arranged with respect to the first movable part 16b. This allows the cell puncture device 10 to achieve high-speed movement of the first movable part 16b when the needle 17a moves at a high speed and punctures the cell S. The cell puncture device 10 can reduce the weight of the actuator, e.g., the piezoelectric element contained in part of the first movable part 16b and reduce a reaction force due to movement of the first movable part 16b, as compared to the case of increasing the size of the piezoelectric element, for example, so that a large force for movement can be obtained in the first movable part 16b when the first movable part 16b receives the reaction force corresponding to the deformation of the vibration damper 19a. As a result, vibration occurring by the reaction force is reduced.

### Second Embodiment

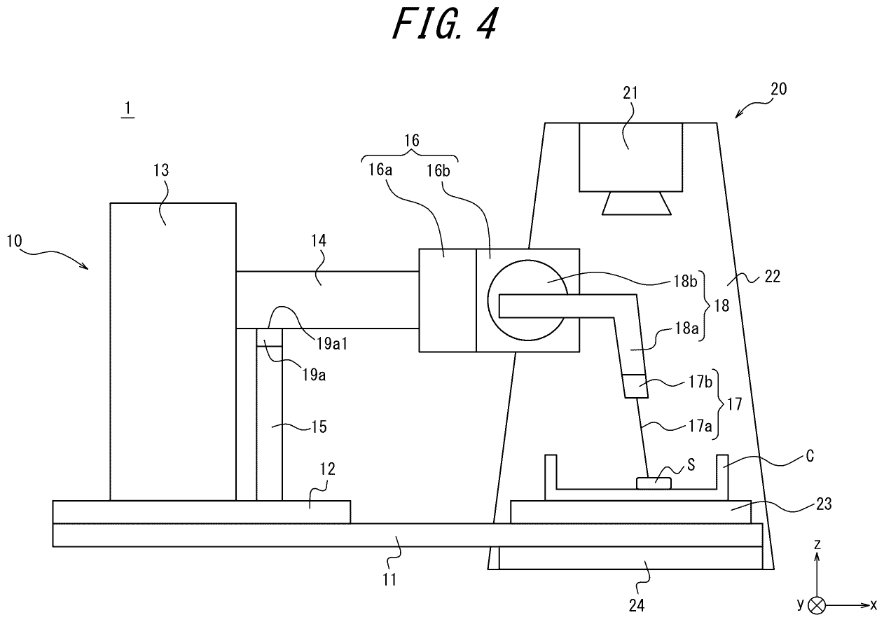

FIG. 4 is a schematic diagram illustrating an example configuration of a microscope system 1 with a cell puncture device 10 according to a second embodiment of the present disclosure. The cell puncture device 10 according to the second embodiment differs from the first embodiment in that the vibration dampers 19a are not arranged with respect to the second driver 16, but the vibration damper 19a is arranged with respect to the arm 14. The other components, functions, effects, variations, and the like are the same as those in the first embodiment, and the corresponding descriptions also apply to the cell puncture device 10 according to the second embodiment. In the following, the same components as in the first embodiment are indicated with the same reference numerals, and descriptions thereof are omitted. The points that differ from the first embodiment will be mainly described.

In the cell puncture device 10 according to the second embodiment, a vibration damper 19a is arranged such that a vibration-damping surface 19al contacts an arm 14. For example, the vibration-damping surface 19al contacts a side surface of the arm 14 that is located on the negative side in the z-direction and that is in parallel with the xy plane.

A second driver 16 is directly connected to the arm 14. The cell puncture device 10 has a support 15 that protrudes from a pedestal 12 in the positive side in the z-direction. The vibration damper 19a is supported at an end of the support 15 that is located on the opposite side to the pedestal 12. Here, the support 15 may be configured, using a slide guide or the like, to be expandable and contractable in the z-direction in accordance with movement of the arm 14. In addition, the support 15 may be configured to be movable in the x- and y-directions and the like by attaching a roller or the like to the bottom of the support 15. Since such a vibration damper 19a connected to the support 15 is in contact with the arm 14, the cell puncture device 10 can suppress vibration of the arm 14.

The cell puncture device 10 according to the second embodiment eliminates the need for arranging the support 15 around the entire second driver 16, and therefore can improve the flexibility of movement of the second driver 16. Therefore, the cell puncture device 10 can suppress the vibration of the needle 17a by arranging the vibration damper 19a on the arm 14 even when a microscope 20 is located near the cell puncture device 10 and the vibration damper 19a is difficult to arrange around the second driver 16. Also, the cell puncture device 10 can dampen vibration that is transmitted to the arm 14, and therefore can suppress vibration that is transmitted to the entire cell puncture device 10 more effectively.

### EXAMPLES

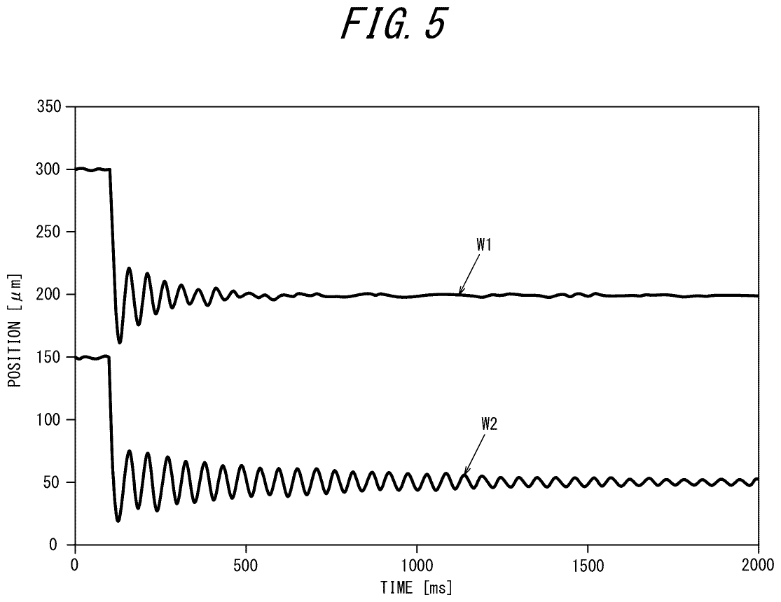

FIG. 5 is a graph for explaining an effect based on the cell puncture device 10 in FIG. 1. The graph in FIG. 5 illustrates two vibration waveforms W1 and W2. The vibration waveform W1 indicates positional variation of the support head 18a with time in association with vibration when the vibration dampers 19a dampen the vibration of the second driver 16 in the cell puncture device 10 in FIG. 1. The vibration waveform W2 indicates, for the purpose of illustrating the effect of the cell puncture device 10 according to the embodiment of the present disclosure, positional variation of the support head 18a with time in association with vibration when the vibration dampers 19a are not provided and the second driver 16 is directly connected to the arm 14. Both the vibration waveforms W1 and W2 indicate the positional variation of the support head 18a with time after the second driver 16 has moved the support head 18a to the negative side in z-direction.

Ideally, the positional variation of the needle 17a with time is desirably illustrated as vibration waveforms, but it is not easy to measure the positional variation of the needle 17a with time due to the fineness of the needle 17a. Accordingly, FIG. 5 illustrates the vibration waveforms W1 and W2 when the positional variation of the support head 18a with time is measured. The needle 17a is firmly fixed to the support head 18a. Therefore, it is conceivable that the positional variation of the needle 17a with time is equivalent to the positional variation of the support head 18a with time.

Both the vibration waveforms W1 and W2 indicate the states of vibration of the support head 18a when the second driver 16 has moved the support head 18a by 100 μm to the negative side in the z-direction. As illustrated in FIG. 5, when the cell puncture device 10 does not hypothetically have the vibration dampers 19a, the vibration waveform W2 indicates that the support head 18a is still vibrating even after 2000 ms (milliseconds) have elapsed. In other words, the vibration is not dampened in a short period of time. On the other hand, when the cell puncture device 10 has the vibration dampers 19a, the vibration dampers 19a dampen the vibration of the support head 18a in a short period of time, and the vibration waveform W1 indicates that the support head 18a is almost not vibrating after 500 ms (milliseconds) have elapsed.

It is obvious to those skilled in the art that the present disclosure can be realized in other predetermined forms excluding the above-described embodiments, without departing from the spirit or the essential features thereof. Therefore, the foregoing descriptions are illustrative and not limited thereto. The scope of the disclosure is defined by the appended claims, not by the foregoing descriptions. Among any changes, some changes within the scope of equivalence thereof shall be included therein.

For example, the shapes, patterns, sizes, arrangements, orientations, types, numbers, and the like of the components described above are not limited to the contents of the descriptions and drawings above. The shapes, patterns, sizes, arrangements, orientations, types, numbers, and the like of the components may be configured arbitrarily as long as the functions thereof can be achieved. The illustrated components of the illustrated cell puncture device 10 and microscope system 1 are conceptual in terms of functions, and the specific forms of the components are not limited to those illustrated.

The functions and the like included in each of the above-described components and the like can be rearranged so that there are no logical contradictions, and it is possible to combine multiple components or the like into one or split one component into multiple.

Examples of some embodiments of the present disclosure are described below. However, it should be noted that the embodiments of the present disclosure are not limited to these examples.

[Appendix 1] A cell puncture device comprising:
a first fixed part arranged with respect to an imaging unit configured to image a cell;
an arm arranged with respect to the first fixed part;
a driver arranged with respect to the arm;
a needle configured to be driven by the driver, the needle configured to puncture the cell; and
a vibration damper configured to dampen vibration of the needle,
wherein the vibration damper is arranged with respect to at least one of the arm or the driver.
   [Appendix 2] The cell puncture device according to appendix 1, wherein the vibration damper is arranged such that a vibration-damping surface intersects a puncture operation direction of the needle.
   [Appendix 3] The cell puncture device according to appendix 1 or 2, further comprising a regulator arranged with respect to the driver, the regulator configured to regulate a movement direction of the driver to a puncture operation direction of the needle.
   [Appendix 4] The cell puncture device according to appendix 3, wherein the regulator includes:
a third fixed part arranged with respect to the arm; and
a second movable part connected to the third fixed part and the driver.
   [Appendix 5] The cell puncture device according to appendix 3 or 4, wherein
the driver includes:
   a second fixed part connected to the regulator; and
   a first movable part connected to the second fixed part, the first movable part configured to drive the needle, and
the vibration damper is arranged such that a vibration-damping surface contacts only the first movable part.
   [Appendix 6] The cell puncture device according to appendix 3 or 4, wherein
the driver includes:
   a second fixed part connected to the regulator; and
   a first movable part connected to the second fixed part, the first movable part configured to drive the needle, and
the vibration damper is arranged such that a vibration-damping surface contacts both the second fixed part and the first movable part.
   [Appendix 7] The cell puncture device according to appendix 3 or 4, wherein
the driver includes:
   a second fixed part connected to the regulator; and
   a first movable part connected to the second fixed part, the first movable part configured to drive the needle, and
the vibration damper is arranged such that a vibration-damping surface contacts only the second fixed part.
   [Appendix 8] The cell puncture device according to any one of appendices 1 to 7, wherein the vibration damper is arranged such that a vibration-damping surface contacts the arm.
   [Appendix 9] The cell puncture device according to any one of appendices 1 to 8, wherein the driver includes a piezoelectric element configured to drive the needle.
   [Appendix 10] The cell puncture device according to any one of appendices 1 to 9, wherein the vibration damper includes vibration-damping rubber.
   [Appendix 11] A microscope system comprising:
the cell puncture device according to any one of appendices 1 to 10; and
a microscope having the imaging unit configured to image the cell to be punctured by the needle of the cell puncture device.

1. A cell puncture device comprising:
a first fixed part arranged with respect to an imaging unit configured to image a cell;
an arm arranged with respect to the first fixed part;
a driver arranged with respect to the arm;
a needle configured to be driven by the driver, the needle configured to puncture the cell; and
a vibration damper configured to dampen vibration of the needle,
wherein the vibration damper is arranged with respect to at least one of the arm or the driver.

2. The cell puncture device according to claim 1, wherein the vibration damper is arranged such that a vibration-damping surface intersects a puncture operation direction of the needle.

3. The cell puncture device according to claim 1, further comprising a regulator arranged with respect to the driver, the regulator configured to regulate a movement direction of the driver to a puncture operation direction of the needle.

4. The cell puncture device according to claim 3, wherein the regulator includes:
a third fixed part arranged with respect to the arm; and
a second movable part connected to the third fixed part and the driver.

5. The cell puncture device according to claim 3, wherein
the driver includes:
a second fixed part connected to the regulator; and
a first movable part connected to the second fixed part, the first movable part configured to drive the needle, and
the vibration damper is arranged such that a vibration-damping surface contacts only the first movable part.

6. The cell puncture device according to claim 3, wherein
the driver includes:
a second fixed part connected to the regulator; and
a first movable part connected to the second fixed part, the first movable part configured to drive the needle, and
the vibration damper is arranged such that a vibration-damping surface contacts both the second fixed part and the first movable part.

7. The cell puncture device according to claim 3, wherein
the driver includes:
a second fixed part connected to the regulator; and
a first movable part connected to the second fixed part, the first movable part configured to drive the needle, and
the vibration damper is arranged such that a vibration-damping surface contacts only the second fixed part.

8. The cell puncture device according to claim 1, wherein the vibration damper is arranged such that a vibration-damping surface contacts the arm.

9. The cell puncture device according to claim 1, wherein the driver includes a piezoelectric element configured to drive the needle.

10. The cell puncture device according to claim 1, wherein the vibration damper includes vibration-damping rubber.

11. A microscope system comprising:
the cell puncture device according to claim 1; and
a microscope having the imaging unit configured to image the cell to be punctured by the needle of the cell puncture device.
